(19) United States
(12) Patent Application Publication
(10) Pub. No.: US 2004/0034342 A1
(43) Pub. Date: Feb. 19, 2004

(54) Medical laser fiber optic cable having improved treatment indicators for BPH surgery

(76) Inventors: Scott N. Barton, Loveland, OH (US); Karen A. Janoff, Lebanon, OH (US); Gregory J. Bakos, Mason, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 09/843,203

(22) Filed: Apr. 26, 2001

(51) Int. Cl. A61B 18/22

(52) U.S. Cl. 606/15; 607/89

(57) ABSTRACT

The present invention relates, in general, to medical laser fibers and, more particularly, to a medical laser fiber optic cable having improved treatment indicators for BPH surgery. Disclosed is a medical instrument for the treatment of tissue comprising a source of light energy; a connector removably attachable to the source of light energy; an optical fiber having a proximal end, connected to the connector, and a distal end positionable at a site of the treatment. The optical fiber comprises: a treatment region, a first depth indicating region, and a second depth indicating region The treatment region includes an active portion and spacer portion. The first depth indicating region originates with a first primary mark at its distal end, terminates with a third primary mark at its proximal end, and includes a second primary mark approximately 5 mm from the first primary mark. A method of gauging the depth of a surgical instrument using one aspect of the present invention comprises the steps of: A) providing a surgical instrument; B) inserting the surgical instrument into tissue; C) viewing at least two non-alphanumeric exposed markings on the surgical instrument, wherein the at least two exposed markings are markedly different markings viewed from a plurality of markings on the surgical instrument, wherein the plurality of markings are arranged such that any two markings will uniquely identify a location on the surgical instrument within a depth indicating region of the surgical instrument; and D) operating the surgical instrument.

### FIELD OF THE INVENTION

The present invention relates, in general, to medical laser fibers and, more particularly, to a medical laser fiber optic cable having improved treatment indicators for BPH surgery.

### BACKGROUND OF THE INVENTION

Currently, surgeons frequently employ medical instruments which incorporate laser technology in the treatment of benign prostatic hyperplasia, or as commonly referred to as BPH. BPH is a condition of an enlarged prostate gland, in which the gland having BPH typically increases in size to between about two to four times from normal. The lasers which are employed by the surgeons to treat this condition must have durable optical fibers that distribute light radially in a predictable and controlled manner.

Typically, surgeons performing BPH surgery with laser fibers use an operating scope. The scope is small enough to insert into the urethra, resulting in a very limited field of view for operating. Precision and control are important to surgeons so that positive clinical outcomes are assured, however the limited view from operating scopes introduces significant difficulty in understanding fiber position and depth.

An optical fiber which is adapted to be employed for this purpose typically contains a glass core surrounded by cladding, a buffer layer, and an outer alignment sleeve. The cladding protects the inherently weaker glass core by imparting a mechanical support to the core. The cladding also ordinarily possesses an index of refraction that is lower than that of the core in order to block light transmitted through the optical fiber from emerging radially from the core.

An optical fiber with a diffuser portion for diffusing light emitted at an end thereof is disclosed in Esch U.S. Pat. No. 5,754,717 as shown in FIG. 1 of this application, which patent is commonly assigned to the present assignee. There is illustrated an optical fiber leading end 10 having a diffuser portion 12 comprised of the stripped core of a typical optical laser, an optical coupling layer, and an outer or alignment sleeve 14. The optical coupling layer, replacing a part of the cladding and the buffer layer of the optical fiber, has an index of refraction exceeding that of the core so as to draw the light out of the core using well-known physical principles. The alignment sleeve is abraded, or roughened, in order to conduct light from the optical coupling layer to the exterior, while heat staking or ultrasonic welding is used to apply or attach the portion of 14a of the outer sleeve 14 covering the diffuser tip to a further separate portion 14b of the sleeve located towards the end of the optical fiber.

U.S. Pat. No. 5,343,543 ('543) discloses a means for marking a laser fiber to reference the direction of light emitted from the tip. The '543 patent teaches in the body a marking system that can be used to determine depth by the amount of marking that remains exposed. Its preferred embodiment is use of a heat shrink marker, but the printing is performed after being shrunk.

The desire for evenly spaced depth markings is difficult when using a heat shrink marker. Because the outer sleeve is made of Teflon, it is difficult to print directly onto the surface. The heat shrink process is difficult to control and consistency of portions of the marker other than the leading edge is lacking. This results in variation, sometimes significant, in the spaces between the printed pattern when a pattern is pre-printed onto the heat shrink material. Therefore, the surgeon is only able to use the leading edge of the marker as an accurate reference point for depth judgement.

U.S. Pat. No. 5,084,022 describes markings that extend around the circumference in the form of bands or other shape marks such as dots. U.S. Pat. No. 4,559,046 mentions a metric scale indicia that can be observed by the surgeon to determine the extent to which the catheter has been inserted. U.S. Pat. No. 5,045,071 describes a depth marking for tracheal tubes that indicates depth by the exposed markings. Normal scales such as, for example, metric rules are too difficult to discern during surgical procedures and have proven to be non-optimal. Regular markings such as, for example, bands or dots suffer from not being able to judge depth easily. Further, the view from an operating scope severely limits both the depth of field and the area of view, making alphanumeric markings difficult to read, and often requiring the surgeon to twist the optical cable to be able to discern the alphanumeric character.

U.S. Pat. No. 3,399,668 describes equally spaced indices on a cholangiography catheter. U.S. Pat. No. 3,605,750 describes an endotracheal tube with three designations along its length for three insertion depths. U.S. Pat. No. 5,004,456 describes visual reference marks for an in-dwelling catheter to indicate insertion depth. U.S. Pat. No. 5,578,018 describes catheter markings of various colors or patterns to determine the length of a stricture in situ.

Although each of the aforementioned laser fibers have attempted to provide laser fibers that were precise and controllable, it would be advantageous to provide an improved laser fiber that is more precise and controllable, where the surgeon can easily discern the treatment position and depth of the light radiating portion of the fiber by visualizing only a short segment of exposed markings, and not all exposed markings. It would also be advantageous to provide a laser fiber that is capable of precise location over a wide range of patient sizes, to accommodate anatomical variation, while maintaining patient safety.

This application is related to the following copending patent application: application Ser. No. 09/785,571 [Attorney Docket No. IND038].

### SUMMARY OF THE INVENTION

The present invention relates, in general, to medical laser fibers and, more particularly, to a medical laser fiber optic cable having improved treatment indicators for BPH surgery. Disclosed is a medical instrument for the treatment of tissue comprising a source of light energy; a connector removably attachable to the source of light energy; an optical fiber having a proximal end, connected to the connector, and a distal end positionable at a site of the treatment. The optical fiber comprises: a treatment region, a first depth indicating region, and a second depth indicating region The treatment region includes an active portion and spacer portion. The first depth indicating region originates with a first primary mark at its distal end, terminates with a third primary mark at its proximal end, and includes a second primary mark approximately 5 mm from the first primary mark. The marks are preferably not alphanumeric characters.

A method of gauging the depth of a surgical instrument using one aspect of the present invention comprises the steps of: providing a surgical instrument; inserting the surgical instrument into tissue; viewing at least two non-alphanumeric exposed markings on the surgical instrument, wherein the at least two exposed markings are markedly different markings viewed from a plurality of markings on the surgical instrument, wherein the plurality of markings are arranged such that any two markings will uniquely identify a location on the surgical instrument within a depth indicating region of the surgical instrument; and operating the surgical instrument.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 illustrates a longitudinal sectional view of an optical fiber utilizing the diffuser portion as shown in the Esch U.S. Pat. No. 5,754,717;

FIG. 2 illustrates a schematic representation of a laser device utilizing the optical fiber;

FIG. 3 illustrates a diagrammatic perspective view of an optical fiber assembly;

FIG. 4 illustrates a longitudinal sectional view of the optical fiber utilizing a diffuser portion, showing as represented from the interior to the exterior thereof, a core, an optical coupling layer, and an outer sleeve contacting the core distal to the diffuser portion;

FIG. 5 illustrates a fragmentary sectional view showing the annulus material containing a light-scattering component;

FIG. 6 illustrates a longitudinal sectional view showing the annulus assembled to the core prior to implementing the tipping step in an optical fiber utilizing the diffuser portion;

FIG. 7 illustrates a longitudinal sectional view of an embodiment of an optical fiber utilizing the diffuser portion showing, as represented from the interior to the exterior, a core, an optical coupling layer, and an outer sleeve contacting the core distal to the diffuser portion;

FIG. 8 illustrates a plan view of the distal end of a medical laser fiber in accordance with the present invention;

FIG. 9 illustrates a perspective view of the distal end of a medical laser fiber in accordance with the present invention where the fiber is viewed as a surgeon might see it through an operating scope; and

FIG. 10 is a flow chart illustrating a method of use of one aspect of the present invention.

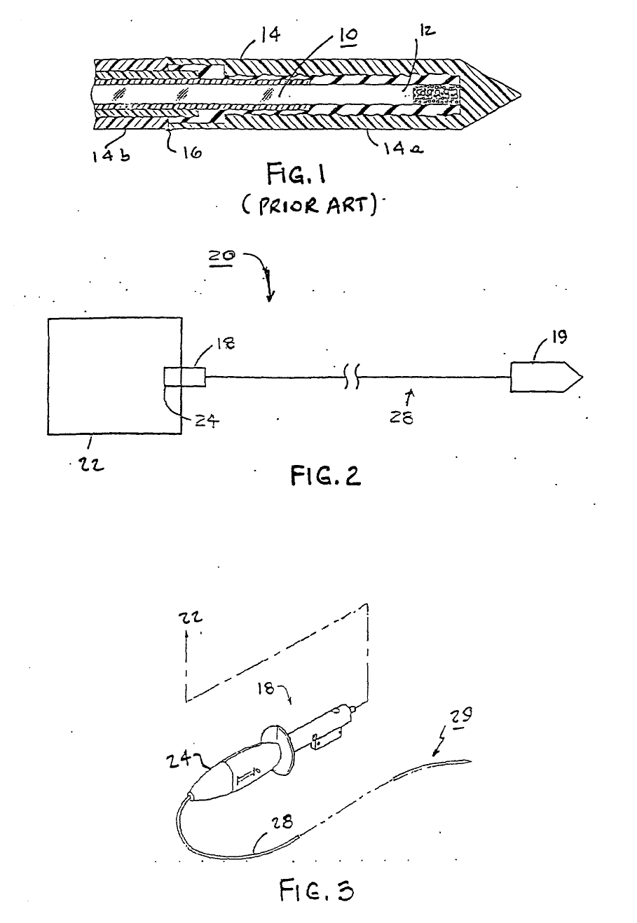

### DETAILED DESCRIPTION OF THE INVENTION

Referring in detail to the drawings, for purposes of this description, and as illustrated in FIG. 2, “proximal” refers to a section on the optical fiber 28 closer to a source of light energy 22, and “distal” refers to a section on the optical fiber which is further away from the source of light energy 22.

For purposes of this description, the term “markedly different” refers to a comparison of markings where the markings have a characteristic or feature that by observation differentiates one marking from an adjacent marking.

Illustrated schematically in FIG. 2 is a medical instrument 20 for diffusing light from an optical fiber 28. The medical instrument 20 includes a source of light energy 22, preferably a laser; and wherein the optical fiber 28 connects into the source of light energy 22 through the intermediary of a connector 18 which is attached to a connection port 24 leading to a diffuser portion 19 of the optical fiber. A typical connector and connection port of this kind which can be utilized for the medical instrument 20 is described in Evans et al. U.S. Pat. No. 5,802,229, while a typical laser employable for the medical instrument 20 is the Optima laser which will be sold by Ethicon Endo-Surgery in Cincinnati, Ohio. The optical fiber 28 with the attached connector 18 can be provided and sold separately from the source of light 22, as an optical fiber assembly 29, as represented in FIG. 3 of the drawings.

A typical optical fiber 28 according to one embodiment of the present invention, includes diffuser portion 19 and a proximal light-transmitting portion 34 is shown in FIG. 4. In a light-transmitting portion 23 of the optical fiber 28, a cladding 32 and the proximal portion 34 of a sleeve 38 radially surround the proximal portion 30 of a core 31. The optical fiber 28 may also have a buffer layer 42 arranged to extend circumferentially between the cladding 32 and the sleeve 38. The material used to form the cladding 32 has an index of refraction lower than the index of refraction of the material used to create the core 31 so as to contain the light within the core 31. The core 31, in addition to its proximal portion 30, extends through a distal portion 36 to the distal end 52 thereof. The distal portion 36 of the core 31 which is employed to diffuse light, is surrounded by an optical coupling layer 40 and the distal portion 44 of the sleeve 38. There is no interruption, discontinuity, or weld joint on the sleeve 38 inasmuch as the proximal portion 34 of the sleeve 38 and the distal portion 44 of the sleeve 38 are two segments of one continuous unitarily constructed sleeve 38. The sleeve 38 can extend distally past the distal end 52 of the core 31 and may be configured to penetrating tip 50. The sleeve 38, as mentioned, is constituted of one continuous piece, preferably consisting of perfluoroalkoxy impregnated with barium sulfate.

A material having an index of refraction higher than the index of refraction of the core 31 forms the optical coupling layer 40, wherein UV50 Adhesive, available from Chemence, Incorporated, in Alpharetta, Ga., can be used to produce the optical coupling layer 40.

A light-scattering component 48 which is filled with a light-scattering material and located on the distal face 52 of the core 31 can reflect light back into the core 31 so as to provide a more even or uniform light distribution, whereby alexandrite can be employed as the light-scattering material for component 48. In addition to its light-scattering properties, the material fluoresces in a temperature-dependent manner upon being stimulated by light, with this property adapted to be used to measure temperature in tissue in proximity to the diffuser portion 19. The same adhesive which is employed for the optical coupling layer 40 can suspend the alexandrite particles therein and can serve as the base material for the light-scattering component 48.

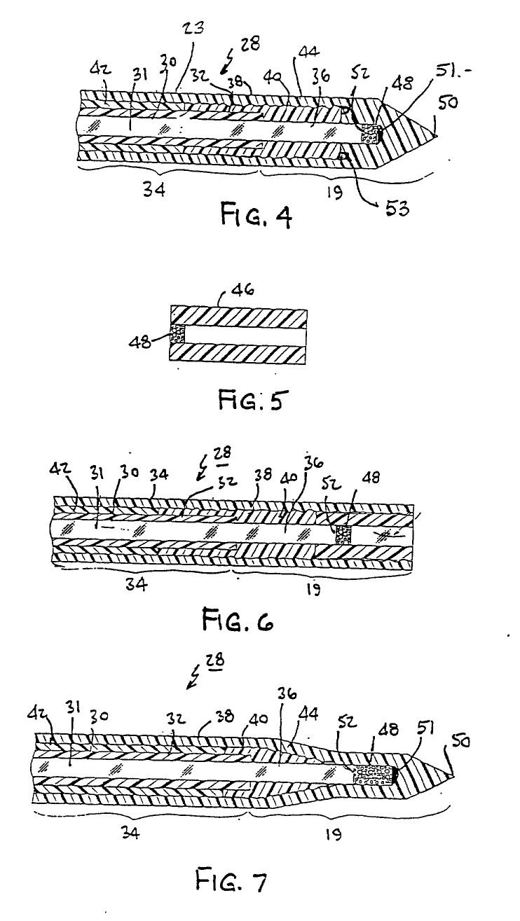

As illustrated in, respectively, FIGS. 4 and 7, utilizing the light-scattering component 48, the sleeve 38 is shaped to extend distally past the light-scattering component 48 and resultingly forms a pointed penetrating tip 50.

During operation of the medical instrument 20, light generated by the source of light energy 22 travels through the core 31 to the diffuser portion 19. There, in the embodiment of the invention illustrated in FIG. 4, light energy emerges from the core 31 to the optical coupling layer 40 because of the optical coupling layer having a higher index of refraction. The distal portion 44 of the sleeve 38 which surrounds the optical coupling layer 40, collects the light from the optical layer 40, employing the abrasions formed on the inner surface of the distal portion 44 of the sleeve 38. The sleeve 38 preferably uses barium sulfate particles scattered within the sleeve 38 to direct light energy evenly outwards towards the tissue. Light energy reaching the light-scattering component 48 is reflected back towards the core 31 by the alexandrite particles in the light-scattering component 48. Moreover, the fluorescent properties of the alexandrite particles, when stimulated by light energy of the proper wavelength, can determine the temperature of surrounding tissues employing methods which are known in the art. The penetrating tip 50 is capable of piercing tough tissue in order to assist medical procedures.

The sleeve 38 has no weld joints or discontinuities in the outer diameter extending from the proximal end of the penetrating tip 50 to the connector 18 which conceivably tend to weaken the optical fiber 28, or which may detrimentally catch or drag the optical fiber 28 so as to displace the latter while in use. When using the optical fiber 28, surgeons or medical practitioners often need to bend it to successfully locate the fiber in the body of a patient. The optical fiber 28 and the associated sleeve 38 can withstand more bending than optical fibers with sleeves which have weld lines or discontinuities formed in the outer diameter thereof proximal to the penetrating tip 50.

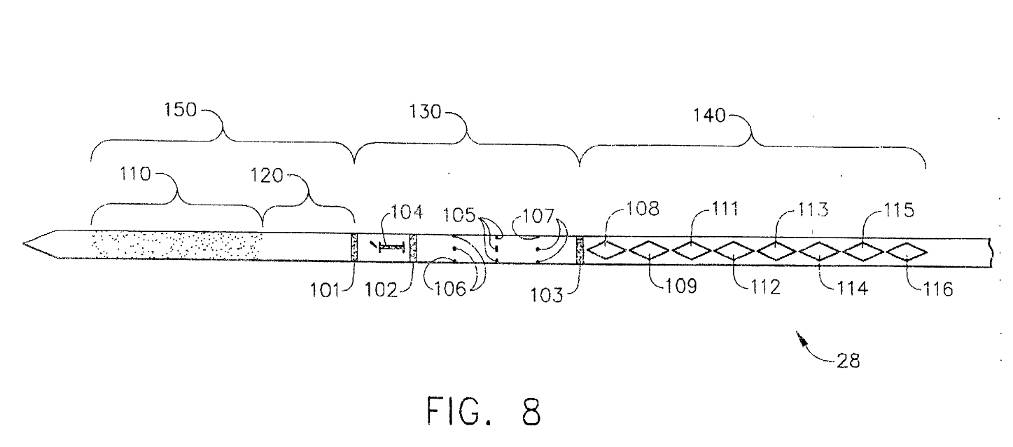

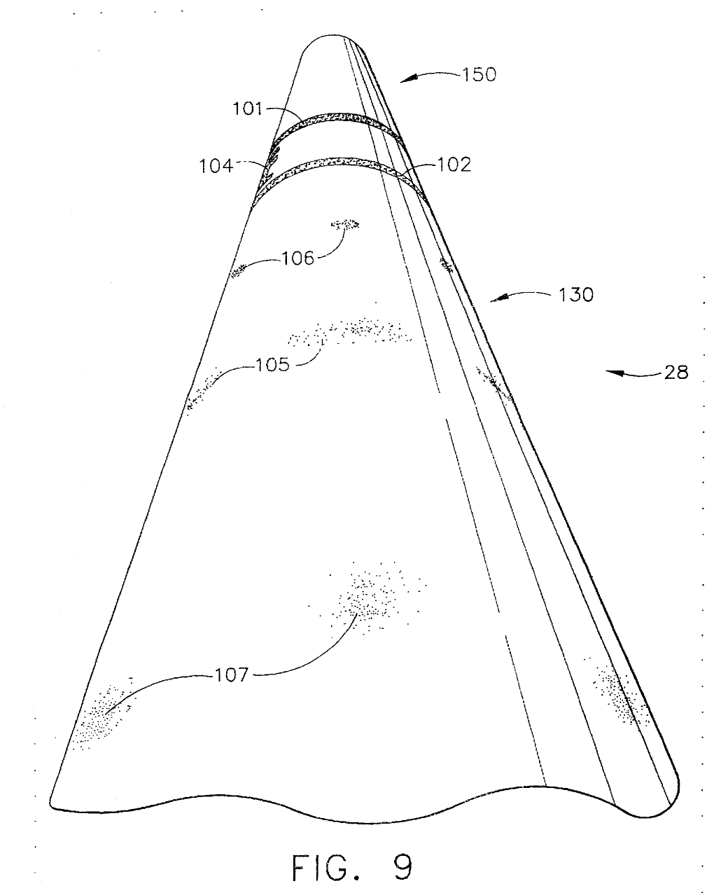

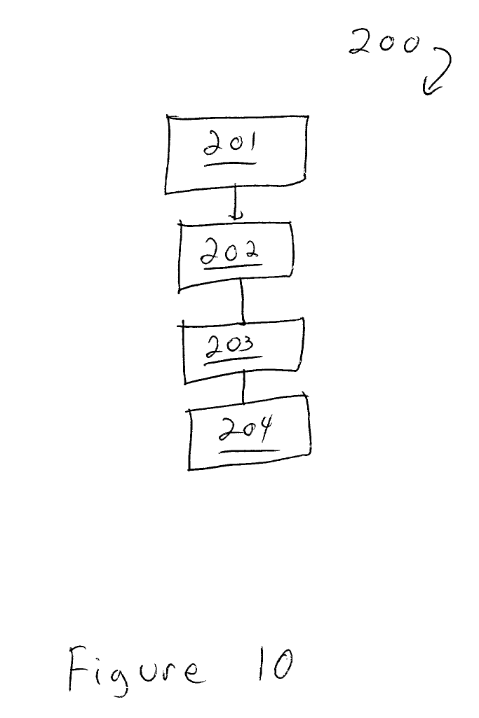

Referring now to FIGS. 8 and 9, optical fiber 28 according to the present invention includes specific markings on optical fiber 28, allowing visual cues at about evenly spaced locations. The surgeon is able to determine depth of optical fiber 28 penetration from any orientation without having to twist the optical fiber 28. The markings are preferably not alphanumeric characters. Preferably markings such as Complete Bands, Dots, Dashes, and Diamonds, are used to segment the distal portion of optical fiber 28 into regions. Markings are preferably on outer sleeve 38.

The different symbols have different meanings to indicate to the surgeon how deep the optical fiber 28 is placed. For example, Complete Bands may indicate preferential (primary) insertion points; Dashes may represent the halfway point between Complete Bands; Dots may represent midway points between Complete Band and Dashes; and Diamonds may represent a depth past the most proximal Complete Band.

Referring to FIG. 8, the distal end of optical fiber 28 includes three regions, a treatment region 150, a first depth indicating region 130, and a second depth indicating region 140. Treatment region 150 includes active portion 110 and spacer portion 120. First depth indicating region 130 originates with a first primary mark 101 at its distal end, terminates with a third primary mark 103 at its proximal end, and includes a second primary mark 102 approximately 5 mm from first primary mark 101 and a main mark 104 between first primary mark 101 and second primary mark 102. Between second primary mark 102 and third primary mark 103 is a halfway point mark 105. Between halfway point mark 105 and third primary mark 103 lies a midway mark 107. Between halfway point mark 105 and second primary mark 102 lies a midway mark 106. Proximal to third primary mark 103, and about evenly spaced, are a series of marks 108, 109, 111, 112, 113, 114, 115, and 116 within second depth indicating region 140. In the example illustrated in FIG. 8, second primary mark 102 and third primary mark 103 are solid lines or complete bands, circumferentially surrounding the complete optical fiber 28, halfway point mark 105 is a set of six dashes (three visible), and midway mark 106 and midway mark 107 are each six dots (three visible).

Treatment region 150 is preferably about 15 mm in length. First depth indicating region 130 is preferably about 14 mm in length. Second depth indicating region 140 is preferably about 20 mm in length. Active portion 110 is preferably about 10 mm in length, and spacer portion 120 is preferably about 5 mm in length. Moving proximally from first primary mark 101 at the distal end, there is preferably a mark at about every 2.5 mm. Within first depth indicating region 130 there will preferably be no repetition of any two sequential marks, such that any two marks are markedly different and can be used to identify a unique location within first depth indicating region 130. First primary mark 101 is preferably positioned about 5 mm from active portion 110. Second primary mark 102 is preferably located about 5 mm proximal to first primary mark 101. Third primary mark 103 is preferably located about 10 mm proximally from second primary mark 102.

As the optical fiber 28 is inserted into tissue to the first depth indication region 130, no two symbols appear in the same order, and therefore either the marking that remains visible or the marking that has been inserted into the tissue can be used to determine depth. By using optimally sized markings with no two in the same order along the length of the fiber, the surgeon can readily discern tip penetration into tissue when only two markings are visible in the operative field of view. This feature is important to control and precision when using laser fibers.

The locations of the primary marks 101, 102 and 103 are intended to make it easy for the surgeon to do the procedure with precision and control. The first primary mark 101 may be used for treatment of median lobes, the end of the second primary mark 102 (proximal to the I) may be used for a single-stick-single-treatment technique, and the third primary mark 103 may be used for a single-stick-double-treatment technique.

The single-stick-single-treatment technique is the typical method of performing an Interstitial Laser Coagulation (ILC) procedure. The optical fiber 28 is punctured through the urethra into the prostate to a depth represented by the second primary mark 102. Once in place, the laser cycle is run to treat the prostate tissue, creating a lesion. After the treatment cycle has concluded, the optical fiber 28 is removed from that puncture site. If another lesion is desired, the optical fiber 28 is punctured to another site and the process begins again.

The single-stick-double-treatment technique is slightly different, and may be applied at the surgeons' discretion. The optical fiber 28 is punctured into the tissue to the second primary mark 102, and the laser cycle is run. After the completion of that cycle, the optical fiber 28 is inserted deeper within the same puncture site to a depth represented by the third primary mark 103. The laser is then run again, resulting in two lesions being formed in series along a single puncture site.

The markings are printed directly onto the optical fiber 28, so the spacing is well controlled. The area between the second primary mark 102 and third primary mark 103 is evenly marked with dotted lines at the quarter-spaces (midway mark 106 and midway mark 107) and a dashed line at the halfway location (halfway point mark 105). The distance from the second primary mark 102 to the third primary mark 103 is preferably about 10 mm. The diamonds, designated series of marks 108, 109, 111, 112, 113, 114, 115, and 116, provide a visual cue that the optical fiber 28 is deep. Series of marks 108, 109, 111, 112, 113, 114, 115, and 116 can also be used for judging penetration depth because each diamond is from one to 5 mm long, and preferably about 2.5 mm long.

The markings are printed on the optical fiber 28 so that they are visible and distinguishable around the entire circumference, from any orientation, to ensure that the surgeon can see them without having to twist the optical fiber 28. This reduces the chance of misinterpreting which marking is being visualized. Utilization of the preferable spacing of 2.5 mm for each marking provides an even continuous scale within the treatment regions 130 and 140 of the optical fiber 28. Markings may be printed, etched, mask-sprayed or otherwise produced by techniques such as, for example, pad printing, or other marking techniques known in the art.

Interrupted marks such as, for example, dots or dashes, preferably have six distinct repetitions around the circumference of optical fiber 28. In FIG. 8, only three of the six dots making midway marks 106 and 107 can be seen. Six repetitions provide a readily discernable mark when viewing any portion of the fiber through a scope. Midway marks 106 and 107 preferably comprise six dots equally spaced circumferentially around optical fiber 28. Halfway point mark 105 preferably comprise six dashes equally spaced circumferentially around optical fiber 28.

The particular marking is not important to the present invention. For example, solid primary marks may be replaced by diamonds, diamonds replaced by solid lines, and dashed lines replaced by dotted lines without departing from the present invention.

FIG. 9 illustrates the utility of the present invention. FIG. 9 illustrates how an optical fiber 28 appears to a surgeon during use, without any tissue obscuring the field of view. For example, during use, optical fiber 28 may be inserted into tissue just past midway marks 106, completely obscuring treatment region 150, first primary mark 101, second primary mark 102, main mark 104, and midway marks 106. Only visible to the surgeon would be halfway point mark 105 and midway marks 107. Because halfway point mark 105 and midway marks 107 are markedly different, and because no two markings are sequentially repeated along the length of optical fiber 28 within treatment region 130, the surgeon knows precisely where the fiber is within the tissue. Also, because the markings are consistent around the entire circumference of the optical fiber 28, the surgeon does not need to twist or rotate the fiber to bring the scale or markings into view.

FIG. 10 is a flow chart illustrating a method of using one aspect of the present invention, designated method 200. Method 200 of gauging the depth of a surgical instrument using one aspect of the present invention comprises the steps of:

A) providing a surgical instrument, designated step 201;

B) inserting the surgical instrument into tissue, designated step 202:

C) viewing at least two non-alphanumeric exposed markings on the surgical instrument, wherein the at least two exposed markings are markedly different markings viewed from a plurality of markings on the surgical instrument, wherein the plurality of markings are arranged such that any two markings will uniquely identify a location on the surgical instrument within a depth indicating region of the surgical instrument, designated step 203; and

D) operating the surgical instrument, designated step 204.

While preferred embodiments of the present invention have been shown and described herein, it will be obvious to those skilled in the art that such embodiments are provided by way of example only. Numerous variations, changes, and substitutions will now occur to those skilled in the art without departing from the invention. Accordingly, it is intended that the invention be limited only by the spirit and scope of the appended claims.

What is claimed is:

1. A medical instrument for the treatment of tissue comprising:
a source of light energy;
a connector removably attachable to said source of light energy;
an optical fiber having a proximal end, connected to said connector, and a distal end positionable at a site of the treatment, said optical fiber comprising:
a treatment region, a first depth indicating region, and a second depth indicating region;
wherein said treatment region includes an active portion and spacer portion;
wherein said first depth indicating region originates with a first primary mark at its distal end, terminates with a third primary mark at its proximal end, and includes a second primary mark approximately 5 mm from said first primary mark.

2. The medical instrument according to claim 1, further comprising a main mark between said first primary mark and said second primary mark.

3. The medical instrument according to claim 2, further comprising a halfway point mark between said second primary mark and said third primary mark.

4. The medical instrument according to claim 3, further comprising a first midway mark between said second primary mark and said halfway point mark; and a second midway mark between said halfway point mark and said third primary mark.

5. The medical instrument according to claim 4, wherein said halfway point mark is markedly different than said first midway mark, and said halfway point mark is markedly different than said second midway mark.

6. A medical instrument for the treatment of tissue comprising:
a source of light energy;
a connector removably attachable to said source of light energy;
an optical fiber having a proximal end, connected to said connector, and a distal end positionable at a site of the treatment, said optical fiber comprising a first depth indicating region;
wherein said first depth indicating region originates with a first primary mark at its distal end, terminates with a second primary mark at its proximal end, and comprises a first mark between said first primary mark and said second primary mark, wherein said first mark is markedly different from said first primary mark, and wherein said first mark is markedly different from said second primary mark.

7. The medical instrument according to claim 6, wherein said first primary mark, said second primary mark, and said first mark are around the entire circumference of said optical fiber.

8. The medical instrument according to claim 7, further comprising a second mark between said first primary mark and said first mark; and a third mark between said first mark and said second primary mark.

9. The medical instrument according to claim 8, wherein said first mark is markedly different than said second mark, and said first mark is markedly different than said third point mark.

10. The medical instrument according to claim 9, wherein said second mark, and said third mark are around the entire circumference of said optical fiber.

11. The medical instrument according to claim 10, wherein said second mark is a plurality of dots, wherein said dots are about equidistantly spaced around the circumference of said optical fiber.

12. The medical instrument according to claim 11, wherein said third mark consists of six dots spaced around the circumference of said optical fiber.

13. The medical instrument according to claim 12, wherein said first mark consists of six dashes spaced around the circumference of said optical fiber.

14. A method of gauging the depth of a surgical instrument comprising the steps of:
A) providing a surgical instrument;
B) inserting said surgical instrument into tissue:
C) viewing at least two non-alphanumeric exposed markings on said surgical instrument, wherein said at least two exposed markings are markedly different markings viewed from a plurality of markings on said surgical instrument, wherein said plurality of markings are arranged such that any two markings will uniquely identify a location on said surgical instrument within a depth indicating region of said surgical instrument; and
D) operating said surgical instrument.

15. A surgical instrument comprising:
an elongated portion having a proximal end and a distal end positionable at a site of use, said elongated portion comprising a first depth indicating region;
wherein said first depth indicating region originates with a first primary mark at its distal end, terminates with a second primary mark at its proximal end, and comprises a halfway point mark between said first primary mark and said second primary mark, wherein said first primary mark, said second primary mark, and said halfway point mark are around the entire circumference of said elongated portion, wherein said halfway point mark is markedly different than said first primary mark, and said halfway point mark is markedly different than said second primary mark, wherein said first primary mark, said second primary mark, and said halfway mark are not alphanumeric characters.

16. The medical instrument according to claim 15, further comprising a first midway mark between said first primary mark and said halfway point mark; and a second midway mark between said halfway point mark and said second primary mark.

17. The medical instrument according to claim 16, wherein said halfway point mark is markedly different than said first midway mark, and said halfway point mark is markedly different than said second midway mark.

18. The medical instrument according to claim 17, wherein said first midway mark, and said second midway mark are around the entire circumference of said elongated portion.

19. The medical instrument according to claim 18, wherein said first midway mark is a plurality of dots, wherein said dots are about equidistantly spaced around the circumference of said elongated portion.

20. The medical instrument according to claim 19, wherein said second midway mark consists of six dots spaced around the circumference of said elongated portion.

21. The medical instrument according to claim 20, wherein said halfway point mark consists of six dashes spaced around the circumference of said elongated portion.

22. A method of use of a surgical instrument comprising the steps of:
A) providing a surgical instrument, said surgical instrument comprising:
an elongated portion having a proximal end and a distal end positionable at a site of use, said elongated portion comprising a first depth indicating region;
wherein said first depth indicating region originates with a first primary mark at its distal end, terminates with a second primary mark at its proximal end, and comprises a halfway point mark between said first primary mark and said second primary mark, wherein said first primary mark, said second primary mark, and said halfway point mark are around the entire circumference of said elongated portion, wherein said halfway point mark is markedly different than said first primary mark, and said halfway point mark is markedly different than said second primary mark;
B) inserting said surgical instrument into tissue; and
C) operating said surgical instrument.
